(19) United States
(12) Patent Application Publication
(10) Pub. No.: US 2026/0325201 A1
(43) Pub. Date: Oct. 22, 2026

(54) MONITORING SYSTEM FACILITATING NEURO-MONITORING AND TISSUE IDENTIFICATION

(71) Applicant: AI BIOMED CORP., Minneapolis, MN (US)

(72) Inventors: Nicholas F. Pergola, Arvada, CO (US); David Hacker, Jacksonville, FL (US); Anirudhan Narasimhan, Plymouth, WI (US)

(21) Appl. No.: 19/725,215

(22) Filed: Jun. 30, 2026

Related U.S. Application Data
(63) Continuation of application No. 17/307,194, filed on May 4, 2021.
(60) Provisional application No. 63/019,605, filed on May 4, 2020.

(51) Int. Cl. A61B 18/14; A61B 18/00; A61B 18/12; A61B 90/13

(52) U.S. Cl. CPC A61B 18/14; A61B 90/13; A61B 2018/00434; A61B 2018/00601; A61B 2018/1253; A61B 2018/126

(57) ABSTRACT

A monitoring system facilitating neuro-monitoring and tissue identification and method for use thereof is provided. The system and method can apply electrical stimulation via a probe to the fibrous tissue and/or the tissue of interest, and can determine a location and/or integrity of nerves or nerve roots therein using stimulated response signals in the fibrous tissue and/or the tissue of interest in response to the electrical stimulation. The system and method also can stimulate the tissue of interest by applying radiation to the tissue of interest from the distal end of the probe, and can identify the tissue of interest using captured radiation from the tissue of interest stimulated by the applied radiation.

### CROSS-REFERENCE TO RELATED APPLICATIONS

This application is a continuation of U.S. patent application Ser. No. 17/307,194, filed on May 4, 2021, which claims the benefit of the filing date of provisional U.S. Patent Application No. 63/019,605, filed on May 4, 2020.

### FIELD

The present technology generally relates to a monitoring system facilitating neuro-monitoring and tissue identification.

### BACKGROUND

Monitoring and identifying soft tissues in a patient is a necessary part of most surgeries. Different surgical instruments have been used to facilitate such monitoring and identifying. For example, surgical instruments monitoring electromyography (EMG) have been used to electrically stimulate soft tissues in order to determine the location and the integrity nerves/nerve roots and the corresponding muscles controlled thereby. The location and the integrity affords identification of nerves and muscles, and the identification affords determinations of whether cutting the identified nerves and muscles is appropriate. Other surgical instruments have been used to identify tissues via stimulating autofluorescence in the tissues, and then identifying tissues so stimulated. The identification of tissues via autofluorescence also affords determinations of whether cutting these tissues is appropriate. Given the limited space of some cavities in a patient and the desire to avoid potential negative consequences of cutting certain tissues during surgery, there is a need for a surgical instrument that can facilitate EMG monitoring and autofluorescent stimulation. Such a surgical instrument can be a surgical probe of which portions thereof can be inserted into a cavity of the patient. Portions of the surgical probe can electrically stimulate the soft tissues, and portions of the surgical probe can stimulate autofluorescence in the soft tissues. The electrical and autofluorescent stimulation can be used to identify the soft tissues, and thereby serve as a guide to whether cutting of the identified tissues is appropriate.

### SUMMARY

In one aspect, the present disclosure provides method of using a patient monitoring system during surgery, the method including inserting a probe of the patient monitoring system into a patient undergoing surgery; using a distal end of the probe to bluntly dissect tissue by separating apart fibrous tissue to gain access to tissue of interest located behind the fibrous tissues; applying electrical stimulation by the probe to the fibrous tissue and/or the tissue of interest; determining a location and/or integrity of nerves or nerve roots therein using stimulated response signals in the fibrous tissue and/or the tissue of interest in response to the electrical stimulation; stimulating the tissue of interest by applying radiation to the tissue of interest from the distal end of the probe; identifying the tissue of interest using captured radiation from the tissue of interest stimulated by the applied radiation, and removing or preserving the tissue of interest during the surgery after identification of the tissue of interest.

In another aspect, the present disclosure provides a method of using a monitoring system during surgery, the method including inserting a probe of the patient monitoring system into a patient undergoing surgery; using a distal end of the probe to bluntly dissect tissue by separating apart fibrous tissue to gain access to tissue of interest located behind the fibrous tissues; applying electrical stimulation by the probe to the fibrous tissue and/or the tissue of interest; determining a location and/or integrity of nerves or nerve roots therein using stimulated response signals in the fibrous tissue and/or the tissue of interest in response to the electrical stimulation; transferring radiation from at least one emitter to the distal end of the probe via at least one optical emitter fiber that extends through at least a portion of the probe to the distal end of the probe; stimulating the tissue of interest by applying the radiation to the tissue of interest from the distal end of the probe; transferring from the distal end of the probe captured radiation from the tissue of interest to at least one detector via at least one optical detector fiber that extends through at least a portion of the probe to the distal end of the probe; identifying by the at least one detector the tissue of interest using the captured radiation from the tissue of interest stimulated by the applied radiation, and removing or preserving the tissue of interest during the surgery after identification of the tissue of interest.

In yet another aspect, the present disclosure provides patient monitoring system using electrical stimulation and radiation to stimulate responses in a patient, the system including a control unit, a probe, at least one emitter, at least one emitter optical fiber, at least one detector, at least one detector optical fiber, and cabling; the control unit being configured to control application of the electrical stimulation through the probe, control operation of the at least one emitter, and control operation of the at least one detector; the probe including a proximal end, an opposite distal end, at least one electrode positioned between the proximal end and the distal end, and the cabling connecting the probe to the control unit, the probe being configured to apply the electrical stimulation to a tissue of interest or tissue adjacent the tissue of interest; the at least one emitter being configured to emit the radiation for stimulating fluorescence in the tissue of interest, and the at least one emitter optical fiber being coupled to the at least one emitter and extending through at least a portion of the probe to the distal end of the probe, the at least one emitter optical fiber being configured to transfer the radiation from the at least one emitter to the distal end of the probe; and the at least one detector being configured to detect the fluorescence from the tissue of interest, and the at least one detector optical fiber being coupled to the at least one detector and extending through at least a portion of the probe to the distal end of the probe, the at least one detector optical fiber being configured to transfer the fluorescence from the distal end of the probe to the at least one detector.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic representation that illustrates portions of a monitoring system according to the present disclosure;

FIG. 2 is a schematic representation that illustrates a patient interface module and a monopolar probe setup that can be used with the monitoring system of FIG. 1;

FIG. 3 is a schematic representation that illustrates the patient interface module and a bipolar probe setup that can be used with the monitoring system of FIG. 1;

FIG. 4 is a schematic representation that illustrates a stimulator module that can be used with the monitoring system of FIG. 1;

FIG. 5 is a schematic representation that illustrates tissue detection module that can be used with the monitoring system of FIG. 1;

FIG. 6 is a side perspective view that illustrates a probe used with that can be used with the monitoring system of FIG. 1;

FIG. 7 is an exploded side perspective view that illustrates the probe of FIG. 6 that can be used with the monitoring system of FIG. 1; and

FIG. 8 is an elevational view of an end of a probe portion that can be used with the probe of FIG. 6.

The details of one or more aspects of the disclosure are set forth in the accompanying drawings and the description below. Other features, objects, and advantages of the techniques described in this disclosure will be apparent from the description and drawings, and from the claims.

### DETAILED DESCRIPTION

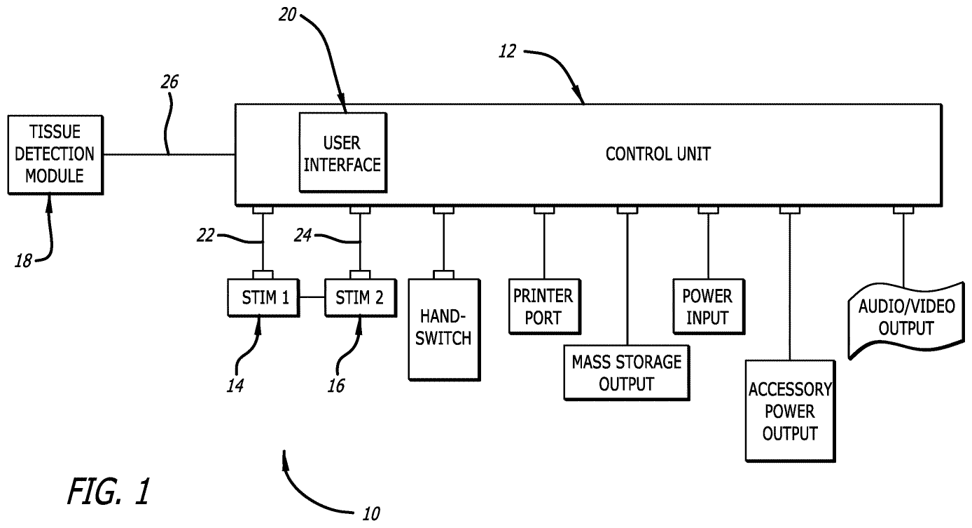

A monitoring system according an embodiment of the present disclosure is generally referenced by the numeral 10 in FIG. 1. The monitoring system 10 incorporates features for facilitating neuro-monitoring (including EMG monitoring) and tissue identification for use in association with a patient P. Features facilitating neuro-monitoring are disclosed in U.S. Pat. Nos. 6,334,068, 7,216,001, and 10,342,452, which are hereby incorporated by reference in their entireties. Such neuro-monitoring can be used to determine neuro integrity. Furthermore, features facilitating tissue identification are disclosed in U.S. Pat. No. 10,579,891 and U.S. application Ser. No. 16/828,803, which are hereby incorporated by reference in its entirety. Such tissue identification can be used to differentiate between different types of tissue.

As depicted in FIG. 1, the monitoring system 10 includes a control unit 12, a patient interface module 14 for being electrically connected with the control unit 12 to deliver Stim 1 electrical stimulation for EMG to the patient P, a stimulator module 16 for being electrically connected with the control unit 12 to deliver Stim 2 electrical stimulation for EMG to the patient P, a tissue detection module 18 for detecting/identifying tissues of the patient P, and a power input for supplying electric power to the control unit 12 from a suitable power source.

Each of the patient interface module 14, the stimulator module 16, and/or the tissue detection module 18 can be separate from or integrated with one another and/or the control unit 12. Furthermore, the monitoring system 10 can include a user interface 20 such as a computer monitor/screen that can be separate from or integrated with the control unit 12, and the control unit 12 can be used to record and/or display data received from the patient interface module 14, the stimulator module 16, and/or the tissue detection module 18. The user interface 20 can, for example, include a touchscreen for facilitating input from a user to control operation of the monitoring system 10, and output of the data resulting from the operation of the monitoring system 10. Suitable user interfaces and the corresponding outputs and inputs thereof are discussed, for example, in U.S. Pat. Nos. 6,334,068 and 7,216,001, and U.S. Ser. No. 16/828,803. Furthermore, the control unit 12 can include a speaker or speakers (not shown) for providing audible communication with the user as discussed, for example, in U.S. Pat. Nos. 6,334,068 and 7,216,001, and U.S. Ser. No. 16/828,803. The control unit 12 may also include a printer port, a mass storage output, an accessory power output and/or an audio/video output as discussed in U.S. Pat. No. 7,216,001.

As depicted in FIG. 1, when not integrated with the control unit 12, the patient interface module 14 can be connected to the control unit 12 via a patient interface connector and wire 22, the stimulator module 16 can be connected to the control unit 12 via a stimulator connector and wire 24, the tissue detection module 18 can be connected to the control unit 12 via a tissue detection connector and a wire or wires 26, and the connectors/wires 22, 24, and 26 may be any suitable electrical connector or wire for delivering power and/or control signals. Use of the connectors/wires 22, 24, and 26 permit the patient interface module 14, the stimulator module 16, and the tissue detection module 18 to be spaced apart from the control unit 12 and positioned adjacent the patient P.

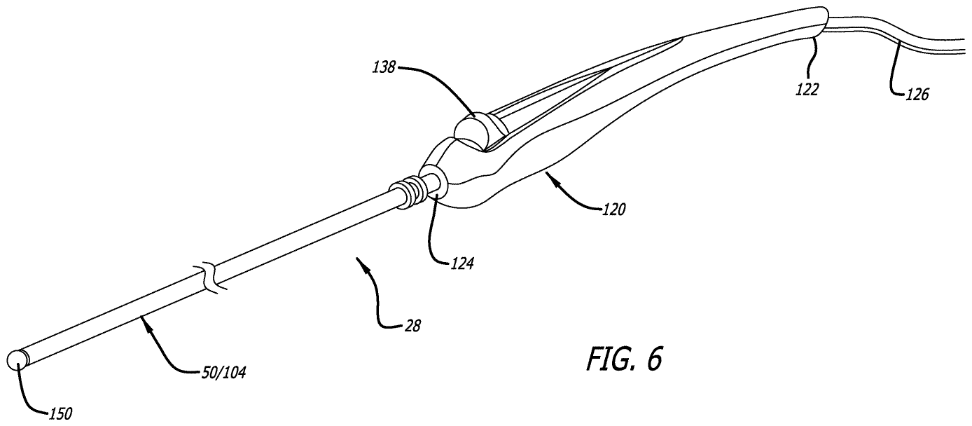

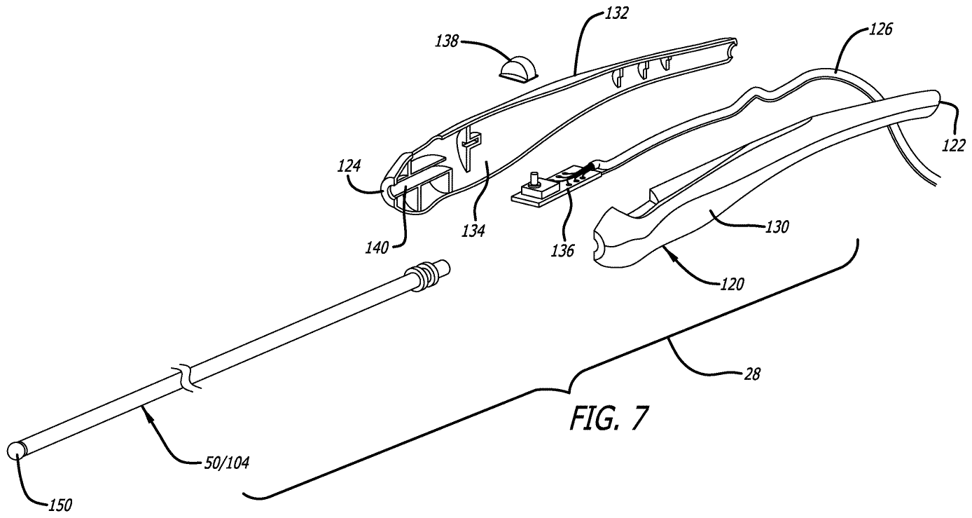

Whether integrated with or separate from the control unit 12, each of the patient interface module 14, the stimulator module 16, and the tissue detection module 18 are connected to a common probe 28 (FIGS. 6 and 7). The probe 28 includes various portions which facilitate operation of the patient interface module 14, the stimulator module 16, and the tissue detection module 18. As discussed below, the interface module 14 can be interconnected with monopolar or bipolar probe portions that are interchangeable with one another and integrated with the probe 28, the stimulator module 16 can be interconnected with a probe portion or portions that are interchangeable with the monopolar or bipolar probe portions and integrated with the probe 28, and the tissue detection module 18 can be interconnected with another probe portion or portions that are integrated with the probe 28 and/or integrated with the other probe portions. Although the probe 28 is shown as having wired connections with the interface module 14, the stimulator module 16, and the tissue detection module 18, the probe 28 could be wirelessly connected to the interface module 16, the stimulator module 16, and/or the tissue detection module 18. Furthermore, the probe 28 can integrate portions of the interface module 16, the stimulator module 16, and/or the tissue detection module 18 to facilitate wireless operation thereof. For example, a wireless connection can connect the probe 28 to the control unit 12.

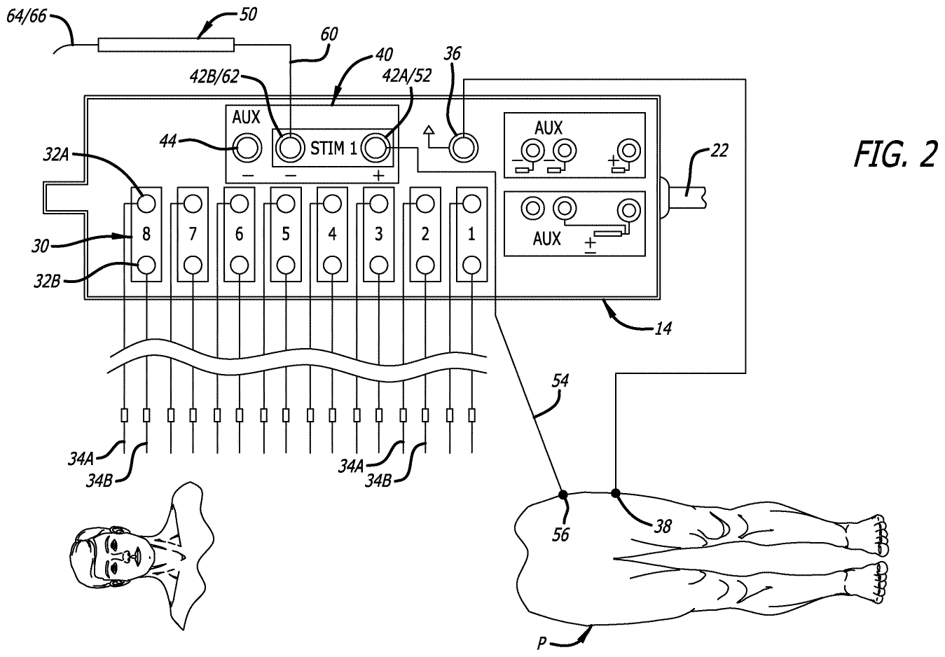

As depicted in FIGS. 2-4, the patient interface module 14 includes a plurality of monitoring channels 30, preferably eight monitoring channels 30, each having two monitoring or recording electrode inputs or connectors (positive and negative) 32A and 32B. The monitoring electrode inputs 32A and 32B are respectively connectible with a corresponding pair of monitoring or recording electrodes (positive and negative) 34A and 34B. The monitoring electrode inputs 32A and 32B may each comprise a jack or other suitable connector for electrical connection with a connector carried at one end of a wire leading from the corresponding monitoring electrode 34A and 34B. The monitoring electrodes 34A and 34B may comprise electrically conductive needles or other suitable structure for insertion in a muscle of the patient P at which EMG activity is to be monitored. The monitoring electrodes 34A and 34B detect EMG activity in the muscles, signals corresponding to the detected EMG activity are transmitted to the control unit 12 via the patient interface module 14, and these signals are displayed as waveforms on the user interface 20 of the control unit 12 as explained in U.S. Pat. Nos. 6,334,068 and 7,216,001. As discussed in U.S. Pat. No. 7,216,001, monitoring EMG activity evoked in response to electrical stimulation provided by the patient interface module 14 allows the location and/or the integrity of nerves and nerve roots (and muscles controlled thereby) to be ascertained.

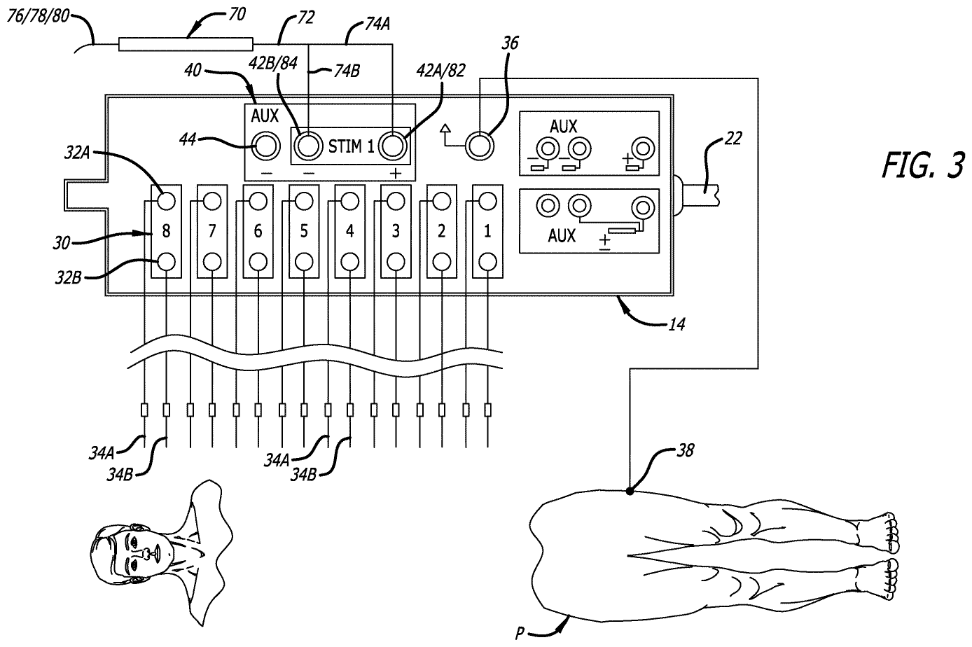

A ground connector 36 is provided on the patient interface module 14 for all monitoring channels 30. As depicted in FIGS. 2 and 3, the ground connector 36 may comprise a jack or other suitable connector for electrical connection with a connector carried at one end of a wire leading from a ground electrode 38. Depending on the intended location for the ground electrode 38 on the patient P, the ground electrode 38 may comprise a conductive needle or any other suitable structure.

As depicted in FIGS. 2 and 3, the patient interface module 14 includes a probe interface 40 for connection of a monopolar or bipolar stimulating probe portion of the probe 28 to the patient interface unit. The probe interface 40 comprises connectors 42A (positive) and 42B (negative) as well as an auxiliary connector 44 (negative). Each probe interface connector 42A, 42B, and 44 may comprise a jack or other suitable electrical connector.

FIG. 2 depicts a representative set-up arrangement for the patient interface module 14 for monopolar Stim 1 stimulation using a monopolar stimulating probe portion 50 of the probe 28. A connector 52 carried at one end of a wire 54 leading from a return electrode (or anode) 56 (positive) is electrically connected with the connector 42A (positive) of the probe interface 40, and the return electrode 56 is applied to the patient P at an appropriate anatomical location. Depending on the intended location for the return electrode 56, the return electrode 56 may comprise single or multiple conductive needles or any other suitable structure for penetrating anatomical tissue. The monopolar probe portion 50 is connected to the probe interface 40 via a connection cable 60 and a connector 62 at the end of the connection cable 60. The connector 62 is electrically connected to the connector 42B (negative), and the monopolar probe portion 50 has a tip 64 comprising an output electrode (or cathode) 66 (negative). The ground electrode 38 is connected to the ground connector 36, and the ground electrode 38 is applied to a non-intervated, electrically neutral anatomical area of the patient P. Pairs of the monitoring electrodes 34A and 34B are connected to the monitoring electrode inputs 32A and 32B, respectively, of a desired number of the monitoring channels 30 for which monitoring is to be conducted. The monitoring electrodes 34A and 34B are inserted in anatomical tissue so as to detect EMG activity in selected musculature.

Stim 1 electrical stimulation in the form of electrical current is delivered to the monopolar probe portion 50 from the patient interface module 14, and the output electrode 66 at the tip 64 delivers monopolar Stim 1 electrical stimulation to anatomical tissue contacted with the output electrode of the tip 64. Electrical current delivered via the monopolar stimulating probe portion 50 flows to the distant return electrode 56, while essentially spreading in all directions from the output electrode at the tip 64. The auxiliary connector 44 can be used if more than one monopolar stimulating probe portion 50 is required to be used during the operative procedure, with both connectors 42B and 44 being controlled by the same stimulation settings selected for Stim 1 electrical stimulation.

FIG. 3 depicts a representative set-up arrangement for the patient interface module 14 for bipoloar Stim 1 electrical stimulation using a bipolar stimulating probe portion 70 of the probe 28. A connection cable 72 leading from the bipolar stimulating probe 70 includes wires 74A and 74B leading from a return electrode (or anode) 76 (positive) and an output electrode (or cathode) 78 (negative), respectively, disposed in close proximity to one another at a tip 80 of the bipolar probe portion 70. A connector 82 at the end of wire 74A is electrically connected with the connector 42A, and a connector 84 at the end of wire 74B is electrically connected with the connector 42B. The monitoring electrodes 34A and 34B and the ground electrode 38 are connected to the patient interface module 14 and applied to the patient P in the same manner as described above for monopolar Stim 1 electrical stimulation. Stim 1 electrical stimulation in the form of electrical current is delivered to the bipolar stimulating probe portion 70 from the patient interface module 14 and, when the tip of the bipolar stimulating probe portion 70 is placed in contact with anatomical tissue, current flows through the tissue directly from the output electrode 78 to the return electrode 76 at the tip 80.

The monopolar probe portion 50 and the bipolar probe portion 70 may be used to provide electrical stimulation in the area of a nerve. If the stimulation is applied at or reasonably near the nerve, the stimulation signal for Stim 1 stimulation is applied to the nerve and is transmitted through the nerve to excite the related muscle. The stimulation signals for Stim 1 stimulation are discussed in detail in U.S. Pat. No. 7,216,001. Excitement of the muscle causes an EMG activity to be generated within the muscle, the impulse being detected by the monitoring electrodes which have been placed in the muscle. Monitoring EMG activity evoked in response to stimulation applied via stimulating probes connected with the patient interface module 14 allows the location and/or integrity of nerves to be ascertained. The monitoring system 10 also allows EMG activity at the monitoring electrodes to be continuously monitored even while no electrical stimulation is being applied and nerves are not being manipulated by the surgeon. Continuous EMG monitoring provides at rest or baseline EMG parameters which facilitate identification of potentially significant intraoperative changes in monitored EMG activity.

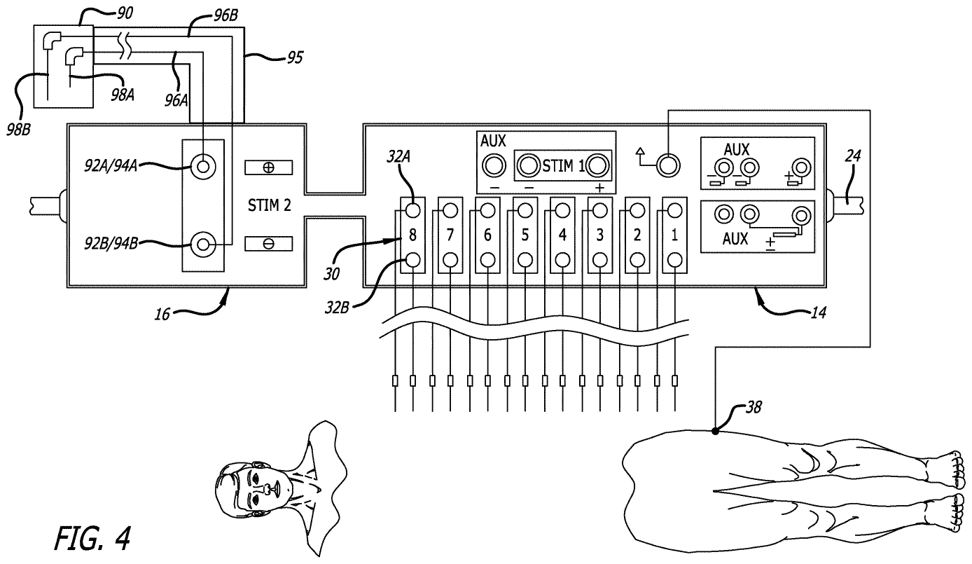

FIG. 4 depicts a representative set-up arrangement for the stimulator module 16 for Stim 2 electrical stimulation using a probe portion 90 of the probe 28. The stimulator module 16 includes two stimulating electrode inputs or connectors 92A and 92B that are an anode (positive) and a cathode (negative), respectively. The stimulating electrode inputs 92A and 92B may each comprise a jack or other suitable electrical connector and are respectively connectable with connectors 94A and 94B carried at an end of a connection cable 95 having wires 96A and 96B leading from a pair of stimulating electrodes 98A and 98B, respectively, as depicted in FIG. 4.

The connector 94A carried at the end of the wire 96A leading from the stimulating return electrode (or anode) 98A (positive) provided on the probe portion 90 is connected to the stimulating electrode input 92A (positive), and the connector 94B carried at the end of the wire 96B leading from the stimulating output electrode (or cathode) 98B (negative) provided on the probe portion 90 is connected to the stimulating electrode input 92B (negative). The patient interface module 14 is arranged with the ground electrode 38 connected and applied as described above for Stim 1 stimulation. Pairs of monitoring electrodes 34A and 34B for a desired number of monitoring channels 30 are connected to the patient interface module 14 and disposed in anatomical tissue to detect EMG activity in musculature affected by the Stim 2 electrical stimulation delivered by the stimulator module 16.

Stim 2 electrical stimulation delivered to the stimulating output electrode 98B flows through the anatomical tissue to the stimulating return electrode 98A. The stimulating electrodes 98A and 98B can be applied to anatomical tissue to be stimulated, and, depending on the intended anatomical location for the stimulating electrodes, the stimulating electrodes 98A and 98B may be configured as part of a probe, low impedance needles, insulated or uninsulated K wires, or any other suitable configuration for penetrating anatomical tissue.

As described in U.S. Pat. No. 7,216,001, depending on the polarity or phase selected for Stim 2 electrical stimulation, the stimulating electrodes 98A and 98B may each function as the output electrode or cathode. For positive phase Stim 2 stimulation, the stimulating electrode 98B functions as the output electrode or cathode with the stimulating electrode 98A functioning as the return electrode or anode. For negative phase Stim 2 stimulation, the stimulating electrode 98A functions as the output electrode or cathode with the stimulating electrode 98B functioning as the return electrode or anode. Such electrical stimulation facilitates the monitoring of EMG activity evoked in response to the electrical stimulation that allows the location and/or the integrity of nerves and nerve roots(and muscles controlled thereby) to be ascertained. The monitoring electrodes detect EMG activity in the muscles, signals corresponding to the detected EMG activity are transmitted to the control unit 12 via the stimulator module 16, and these signals are displayed as waveforms on the user interface 20 of the control unit 12 as explained in U.S. Pat. Nos. 6,334,068 and 7,216,001.

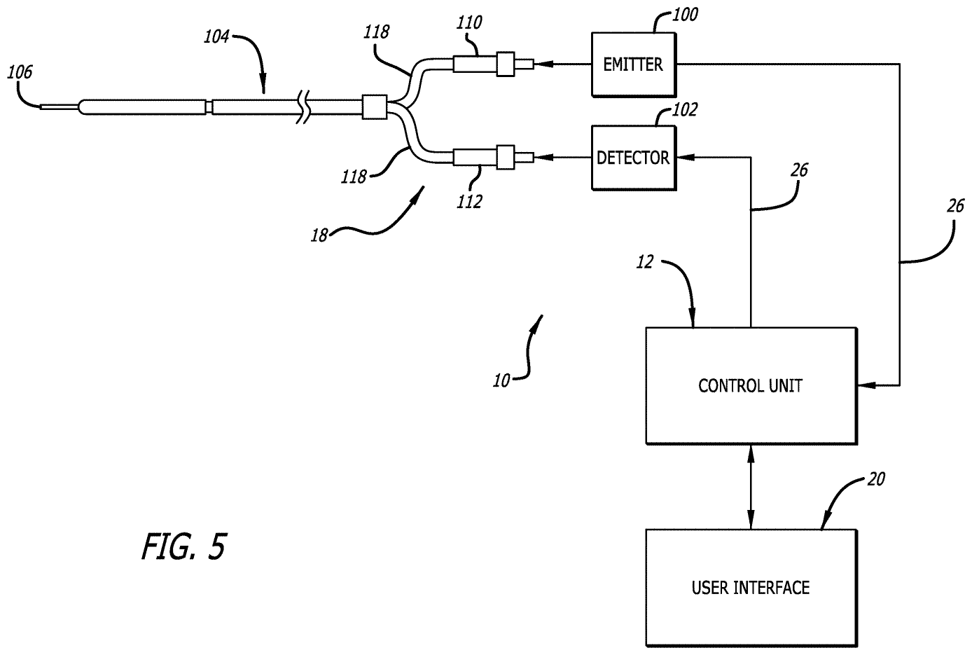

FIG. 5 depicts a representative set-up arrangement for the tissue detection module 18. The tissue detection module 18 can include one or more emitters 100, one or more detectors 102, and a probe portion 104, as described in U.S. Ser. No. 16/828,803. The control unit 12 and the user interface 20 can be used to control operation of the emitter(s) 100, the detector(s) 102, and the probe portion 104 in similar fashion to that described in U.S. Ser. No. 16/828,803.

As discussed in U.S. Ser. No. 16/828,803, the radiation from the emitter 100 can be applied to a tissue of interest to stimulate auto-fluorescence, and the detector 102 can be used to capture the resulting auto-fluorescence in the tissue of interest. The emitter 100 can be a solid state laser or a laser diode. The emitter 100 can be configured to emit radiation at a selected wavelength to stimulate fluorescence, the detector 102 can be configured to process radiation captured by the probe portion 104, and the probe portion 104 can be used to facilitate such stimulation and detection.

The emitter 100 and the detector 102 can be separate from or part of the probe portion 104. When the emitter 100 and the detector 102 are separate from the probe portion 104, the probe portion 104 can include a distal end 106, where portions of an emitter optical fiber or fibers 110 connected to the emitter 100 can extend through portions of the probe portion 104 to the distal end 106, and portions of a detector optical fiber or fibers 112 connected to the detector 102 can extend through portions of the probe portion 104 to the distal end 106 of the probe portion 104. Alternatively, the probe portion 104 can incorporate the emitter 100 and/or the detector 102 at or adjacent the distal end thereof, and the need for lengths of the emitter optical fiber or fibers 110 and/or lengths of the detector optical fiber or fibers 112 extending through the probe portion 104 can thereby be reduced or eliminated. Furthermore, an additional camera such as those described in U.S. Ser. No. 16/828,803 can also be used as detectors.

Portions of the emitter optical fiber or fibers 110 and/or the detector optical fiber or fibers 112 can extend through a connection cable or cables 118 that extend between the emitter 100 and/or the detector 102, and the probe 28 and the probe portion 104. The connection cable or cable 118 can also be used to transfer control signals from the probe 28 and the probe portion 104 to the emitter 100 and/or the detector 102 to control operation thereof.

Similarly to U.S. Ser. No. 16/828,803, the control unit 12 can be used to control the transmission of the radiation from the emitter 100 and to control the detection of the radiation at the detector 102. The connectors/wires 26 can connect the tissue detection module 18, and by connecting the emitter 100 and the detector 102 to the control unit 12. In use, the emitter 100 (via control using the control unit 12 and the user interface 20) along with optical element(s) 114 (such as one of optical lenses and/or filters or the like) is configured to deliver radiation chosen to illuminate in order to stimulate fluorescence through the emitter optical fiber or fibers 110 to the distal end 106 of the probe portion 104. And in use, the detector 102 (via control using the control unit 12 and the user interface 20) along with optical element(s) 116 are configured to detect radiation collected at the distal end 106 of the probe portion 104 through the detector optical fiber or fibers 112. The optical element(s) 114 and 116 can be filters provided at the distal end 106, but other arrangements for filtering in the fiber coupling, or emitters and detectors themselves are possible. The user interface 20 can be used as a display for showing results of the use of the probe portion 104 in similar fashion to that disclosed in U.S. Ser. No. 16/828,803.

The probe 28 can integrate one or more of the probe portion 50, the probe portion 70, the probe portion 90, and the probe portion 104. These probe portions can be interchangeable with one another or be permanent portions of the probe 28. To illustrate, the probe portion 50, the probe portion 70, and the probe portion 90 can be interchangeable with one another in the probe 28, so that the user can change the type of probe portion to facilitate a corresponding operation thereof. As discussed below, for example, the probe portion 50 (which is monopolar) and the probe portion 70 (which is bipolar) can be interchanged with one another.

The probe 28 can include features of a probe assembly disclosed in U.S. Pat. No. 10,342,452. As depicted in FIGS. 6 and 7, the probe 28 can include a probe handle 120 having a proximal end 122 and a distal end 124. All or portions of the connection cable 60 for the probe portion 50, all or portions of the connection cable 72 for the probe portion 70, all or portions of the connection cable 95 for the probe portion 90, and all or portions of the connection cable or cables 118 for the probe portion 104 can be combined together as cabling 126 that can extend through the proximal end 122 and terminate within the probe handle 120 (FIG. 7).

As depicted in FIGS. 6 and 7, the probe handle 120 includes a first handle portion 130 and a second handle portion 132 that are attached to one another and that define a probe interior 134. The probe handle 120 can include circuitry 136 provided in the probe interior 134 to which portions of the cabling 126 is connected to control operation of the probe portion 50, the probe portion 70, the probe portion 90, and/or the probe portion 104. Furthermore, a switch or switches 138 can be included on the probe handle 120 that are operatively connected (via wired connection(s) and/or wireless connection(s)) to the circuitry 136, the control unit 12, the patient interface module 14, the stimulator module 16, and/or the tissue detection module 18. The switch or switches 138 via use of the circuitry 136 and/or the cabling 126 can be used to initiate and terminate operation of the probe portion 50, the probe portion 70, the probe portion 90, and the probe portion 104 through control of the control unit 12, the patient interface module 14, the stimulator module 16, and/or the tissue detection module 18.

As depicted in FIG. 7, the probe 28 can also include a probe connector 140 provided at the distal end 124 of the probe handle 120 that facilitates interchangeability of the probe portion 50 of the patient interface module 14, the probe portion 70 of the patient interface module 14, and the probe portion 90 of the stimulator module 16. The probe connector 140 can be a quick-change connector for securely attaching and releasing end portions of the probe 50, the probe portion 70, and the probe portion 90. To that end, the probe connector 140 can include a button that can facilitate engagement and/or disengagement thereof to facilitate attachment and/or release of the probe portion 50, the probe portion 70, and the probe portion 90. As such, depending on the desired operation of the probe 28, a desired one of the probe portion 50, the probe portion 70, and the probe portion 90 can be used. Furthermore, the control unit 12 and the user interface 20 can be configured to recognize which one of the probe portion 50, the probe portion 70, and the probe portion 90 are attached to the probe 28 via use of the cabling 126 and the circuitry 136. FIGS. 6 and 7 depict the probe portion 50 being used with the probe 28.

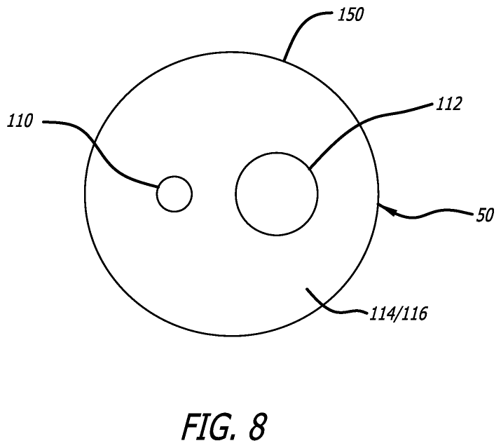

The probe portion 104 of the tissue detection module 18 also can be configured to be interchangeable with the probe portion 50, the probe portion 70, and/or the probe portion 90 using the probe connector 140. Furthermore, a separate probe portion 104 can alternatively be used with the desired one of the probe portion 50, the probe portion 70, and the probe portion 90 via use of a second probe connector (not shown) provided at the distal end 124 of the probe handle 120. Using the second probe connector, the probe portion 104 can be positioned adjacent the desired one of the probe portion 50, the probe portion 70, and the probe portion 90. The probe portion 104 can also alternatively be integrated into the probe portion 50, the probe portion 70, and/or the probe portion 90. When integrated therewith, the emitter optical fiber or fibers 110 and the detector optical fiber or fibers 112 can extend through at least portions of the probe portion 50, the probe portion 70, and the probe portion 90. The emitter optical fiber or fibers 110 and the detector optical fiber or fibers 112 can terminate at corresponding distal ends of the probe portion 50, the probe portion 70, and the probe portion 90, respectively, and the optical element(s) 114 and 116 can be provided at the corresponding distal ends (e.g., the distal end 150 of the probe portion 50). As depicted in FIGS. 6 and 7, the probe portion 104 is integrated with the probe portion 50, and the emitter optical fiber or fibers 110 and the detector optical fiber or fibers 112, as depicted in FIG. 8 terminate at a distal end 150 of the probe portion 50. Alternatively, the probe portion 50, the probe portion 70, and the probe portion 90 can incorporate the emitter 100 and/or the detector 102 at or adjacent the distal ends thereof (e.g., the distal end 150 of the probe portion 50).

While the probe portion 104 can be interchangeable and/or combined with the probe portion 50, the probe portion 70, and the probe portion 90, the probe portion 104 (and other portions of the tissue detection module 18) can be used exclusively with the probe 28 to facilitate tissue detection. The switch or switches 138 included on such a probe 28 can be operatively connected (via wire connection(s) and/or wireless connection(s)) to the circuitry 136, the control unit 12, the probe portion 104, and/or the other portions of the tissue detection module 18. As such, the switch or switches 138 can be used to initiate and terminate operation of the probe portion 104 (and the other portions of the tissue detection module 18) incorporated in such a probe 28 via use of the circuitry 136 and/or the cabling 126.

During use in surgery, the probe portion 50 of the patient interface module 14, the probe portion 70 of the patient interface module 14, the probe portion 90 of the stimulator module 16, and the probe portion 104 of the tissue detection module 18 can be positioned at and adjacent tissues of interest. In doing so, portions of the probe portion 50, the probe portion 70, the probe portion 90, and the probe portion 104 are contacted to the tissues of interest in similar fashion to that described in U.S. Pat. Nos. 7,216,001 and 10,342,452 and U.S. Ser. No. 16/828,803. The user can then initiate operation of the probe portion 50, the probe portion 70, the probe portion 90, and/or the probe portion 104 using the switch or switches 138 or other componentry in accordance with U.S. Pat. Nos. 6,334,001, 7,216,001, and 10,342,452 and U.S. Ser. No. 16/828,803.

At the same time probe portion 50, the probe portion 70, and the probe portion 90 are being operated, operation of the probe portion 104 (when separately used with or integrated into the probe portion 50, the probe portion 70, and the probe portion 90) can also be initiated using the switch or switches 138. When using the separate probe portion 104, a distal end (not shown) of the separate probe portion 104 can be brought into contact or near contact (i.e., within at least 1-2 cm) with potentially fluorescing materials of a region of interest within the body. Furthermore, when using the probe portion 50, the probe portion 70, and the probe portion 90 with the integrated probe portion 104, the corresponding distal ends (e.g., the distal end 150) can also be brought into contact or near contact (i.e., within at least 1-2 cm) with the potentially fluorescing materials of the region of interest in the body. Using the switch or switches 138 or other componentry in accordance with U.S. Ser. No. 16/828,803, operation of the separate or integrated probe portion 104 can then be initiated by the control unit 12 and/or the tissue detection module 18 to emit radiation from the emitter 100 to stimulate auto-fluorescence in the tissue of interest, and to capture by the detector 102 of the resulting auto-fluorescence in the tissue of interest.

When using the tissue detection module 18, a detected fluorescence signal for a tissue of interest, as discussed in U.S. Ser. No. 16/828,803, is compared to a threshold fluorescence signal for a reference tissue to determine if the detected fluorescence signal is indicative of the presence of the reference tissue. There may be an audio and/or visual indication that suitable fluorescence signal has been detected using the control unit 12 and the user interface 20. Because the distal end 106 of the probe portion 104 or the corresponding distal ends (e.g., the distal end 150) of the probe portion 50, the probe portion 70, and the probe portion 90 are small, the emitter optical fiber or fibers 110 and/or the detector optical fiber or fibers 112 terminate in a small area at these distal ends, or the emitter 100 and/or the detector 102 are provided at or adjacent the small area at these distal ends, and because this small area is in contact or near contact with the surface of the tissue of interest, the area exposed to illumination/stimulation and detection is quite small, thereby allowing for a precise location of the tissue of interest. The identification of tissues by the tissue detection module can be made using an affirmative or negative identification of the tissue. Once the tissue of interest is identified, the tissue of interest can be removed or preserved during surgery. For example, parathyroid material, thyroid material, and other tissues in the neck region can be identified to facilitate removal or preservation during surgery using the tissue detection module 18.

The probe portion 50, the probe portion 70, the probe portion 90, and the probe portion 104, whether or not the probe portion 104 is integrated therewith, can be used in blunt dissection of the tissue of interest by contacting the distal ends thereof with tissues that can be separated via contact with the distal ends of the probe portions. To illustrate, skeletal muscles are fibrous, and the strands of muscle fibers can be separated using the distal ends of the probe portion 50, the probe portion 70, the probe portion 90, and the probe portion 104. Thus, when operating on a patient's neck, for example, the distal ends of the probe portion 50, the probe portion 70, the probe portion 90, and the probe portion 104 can be used in separating the neck muscles to gain access to the thyroid and the parathyroid of the patient. In other words, the distal ends of the probe portion 50, the probe portion 70, the probe portion 90, and the probe portion 104 can be inserted between various fibers of the neck muscles to force separation therebetween to provide access to the thyroid and parathyroid of the patient.

During such blunt dissection, the probe 28 can be operated. The probe portion 50, the probe portion 70, and the probe portion 90 can electrically stimulate, for example, the above-discussed neck muscles to facilitate the monitoring of EMG activity evoked in response to such electrical stimulation that allows the location and/or the integrity of nerves and nerve roots in the neck to be ascertained. Furthermore, the probe portion 104 can be operated in conjunction with the probe portion 50, the probe portion 70, and the probe portion 90 to facilitate tissue identification. Operation of the probe portion 104 can occur parallelly or serially with operation of the probe portion 50, the probe portion 70, and the probe portion 90. For example, when the probe portion 104 is integrated with the probe portion 50, the probe portion 70, and the probe portion 90, these probes can be operated simultaneously to determine the location and the integrity of the nerves and the nerve roots, and to identify tissue adjacent the distal ends of the integrated probe portion 50, the probe portion 70, and the probe portion 90.

The user interface 20 can also be used in conjunction with the switch or switches 138 to control operation of the monitoring system 10. For example, the user interface 20 can be a touchscreen facilitating user interaction to configure operation of the control unit 12, the patient interface module 14, the stimulator module 16, and/or the tissue detection module 18.

As discussed above, the monitoring electrodes detect EMG activity in the muscles, signals corresponding to the detected EMG activity are transmitted to the control unit 12 via the patient interface module 14 and the stimulator module 16, and these signals are displayed as waveforms on the user interface 20 of the control unit 12 as explained in U.S. Pat. Nos. 6,334,068 and 7,216,001. Furthermore, the user interface 20 can display the results of the use of the probe portion 104 and the tissue detection module 18 in similar fashion to that disclosed in U.S. Ser. No. 16/828,803

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A method of using a patient monitoring system during surgery, the method comprising:
inserting a probe of the patient monitoring system into a patient undergoing surgery;
using a distal end of the probe to bluntly dissect tissue by separating apart fibrous tissue to gain access to tissue of interest located behind the fibrous tissues;
applying electrical stimulation by the probe to the fibrous tissue and/or the tissue of interest;
determining a location and/or integrity of nerves or nerve roots therein using stimulated response signals in the fibrous tissue and/or the tissue of interest in response to the electrical stimulation;
stimulating the tissue of interest by applying radiation to the tissue of interest from the distal end of the probe;
identifying the tissue of interest using captured radiation from the tissue of interest stimulated by the applied radiation, and
removing or preserving the tissue of interest during the surgery after identification of the tissue of interest.

2. The method of claim 1, wherein the probe is one of a monopolar probe and a bipolar probe.

3. The method of claim 2, further comprising positioning monitoring electrodes on the patient to receive the stimulated response signals.

4. The method of claim 1, wherein the monitoring system includes at least one of an emitter and an emitter optical fiber, the at least one emitter being separate from or integrated with the probe, and being configured to emit the radiation for stimulating fluorescence in the tissue of interest, and the at least one emitter optical fiber being coupled to the at least one emitter and extending through at least a portion of the probe to the distal end of the probe when the emitter is separate from the probe; and
further comprising transferring the radiation from the at least one emitter to the distal end of the probe via the at least one emitter optical fiber to facilitate stimulation of the fluorescence in the tissue of interest by the at least one emitter.

5. The method of claim 4, wherein the at least one emitter comprises a solid state laser or a laser diode.

6. The method of claim 4, wherein a filter is used to limit emitter bandwidth from the at least one emitter.

7. The method of claim 1, wherein the captured radiation is fluorescence from the tissue of interest;
wherein the monitoring system includes at least one of a detector and a detector optical fiber, the at least one detector being separate from or integrated with the probe, and being configured to detect the fluorescence from the tissue of interest, and the at least one detector optical fiber being coupled to the at least one detector, and extending through at least a portion of the probe to the distal end of the probe when the detector is separate from the probe; and
further comprising transferring the fluorescence from the distal end of the probe to the at least one detector via the at least one optical detector optical fiber to facilitate identification by the at least one detector.

8. The method of claim 7, wherein the at least one detector is a near infrared camera with a highpass filter, and wherein the highpass filter is configured to pass optical wavelengths above emitted wavelengths of the radiation used to stimulate the tissue of interest.

9. A method of using a monitoring system during surgery, the method comprising:
inserting a probe of the patient monitoring system into a patient undergoing surgery;
using a distal end of the probe to bluntly dissect tissue by separating apart fibrous tissue to gain access to tissue of interest located behind the fibrous tissues;
applying electrical stimulation by the probe to the fibrous tissue and/or the tissue of interest;
determining a location and/or integrity of nerves or nerve roots therein using stimulated response signals in the fibrous tissue and/or the tissue of interest in response to the electrical stimulation;
transferring radiation from at least one emitter to the distal end of the probe via at least one optical emitter fiber that extends through at least a portion of the probe to the distal end of the probe;
stimulating the tissue of interest by applying the radiation to the tissue of interest from the distal end of the probe;
transferring from the distal end of the probe captured radiation from the tissue of interest to at least one detector via at least one optical detector fiber that extends through at least a portion of the probe to the distal end of the probe;
identifying by the at least one detector the tissue of interest using the captured radiation from the tissue of interest stimulated by the applied radiation, and
removing or preserving the tissue of interest during the surgery after identification of the tissue of interest.

10. The method of claim 9, wherein the probe is one of a monopolar probe and a bipolar probe.

11. The method of claim 9, further comprising positioning monitoring electrodes on the patient to receive the stimulated response signals.

12. The method of claim 9, wherein fluorescence is stimulated in the tissue of interest using the radiation, and the captured radiation is the fluorescence stimulated in the tissue of interest.

13. The method of claim 9, wherein the at least one emitter comprises a solid state laser or a laser diode; and wherein a filter is used to limit emitter bandwidth from the at least one emitter.

14. The method of claim 9, wherein the at least one detector is a near infrared camera with a highpass filter, and wherein the highpass filter is configured to pass optical wavelengths above emitted wavelengths of the radiation used to stimulate the tissue of interest.

15. A patient monitoring system using electrical stimulation and radiation to stimulate responses in a patient, the system comprising:
a control unit, a probe, at least one emitter, at least one emitter optical fiber, at least one detector, at least one detector optical fiber, and cabling;
the control unit being configured to control application of the electrical stimulation through the probe, control operation of the at least one emitter, and control operation of the at least one detector;
the probe including a proximal end, an opposite distal end, at least one electrode positioned between the proximal end and the distal end, and the cabling connecting the probe to the control unit, the probe being configured to apply the electrical stimulation to a tissue of interest or tissue adjacent the tissue of interest;
the at least one emitter being configured to emit the radiation for stimulating fluorescence in the tissue of interest, and the at least one emitter optical fiber being coupled to the at least one emitter and extending through at least a portion of the probe to the distal end of the probe, the at least one emitter optical fiber being configured to transfer the radiation from the at least one emitter to the distal end of the probe; and
the at least one detector being configured to detect the fluorescence from the tissue of interest, and the at least one detector optical fiber being coupled to the at least one detector and extending through at least a portion of the probe to the distal end of the probe, the at least one detector optical fiber being configured to transfer the fluorescence from the distal end of the probe to the at least one detector.

16. The patient monitoring system of claim 15, wherein the patient monitoring system is configured to identify the tissue of interest using the fluorescence detected by the at least one detector.

17. The patient monitoring system of claim 15, wherein the probe is one of a monopolar probe and a bipolar probe.

18. The patient monitoring system of claim 15, wherein the at least one emitter comprises a solid state laser or a laser diode; and wherein a filter is used to limit emitter bandwidth from the at least one emitter.

19. The patient monitoring system of claim 15, wherein the at least one detector is a near infrared camera with a highpass filter, and wherein the highpass filter is configured to pass optical wavelengths above emitted wavelengths of the radiation used to stimulate the tissue of interest.

20. The patient monitoring system of claim 15, further comprising transferring the captured radiation from the distal end of the probe to the at least one detector via the at least one optical detector optical fiber to facilitate identification by the at least one detector.
